(19) United States
(12) Patent Application Publication
(10) Pub. No.: US 2026/0297488 A1
(43) Pub. Date: Oct. 1, 2026

(54) BIOREACTOR WITH NOZZLE MIXING

(71) Applicant: GEA LIQUID TECHNOLOGIES GERMANY GMBH, Sarstedt (DE)

(72) Inventors: Jens Dahl Kunoy, Skanderborg (DK); Daniel Grenov, Skanderborg (DK); Emil Munch Quist, Skanderborg (DK)

(73) Assignee: GEA LIQUID TECHNOLOGIES GERMANY GMBH, Sarstedt (DE)

(21) Appl. No.: 19/514,673

(22) PCT Filed: Aug. 30, 2024
(86) PCT No.: PCT/EP2024/074340

§ 371 Date: Feb. 26, 2026

(30) Foreign Application Priority Data
Aug. 31, 2023 (EP) 23194627.8

(51) Int. Cl. C12M 1/00; C12M 1/02

(52) U.S. Cl. CPC C12M 29/06; C12M 27/00; C12M 29/18

(57) ABSTRACT

The present invention relates to a bioreactor (1) comprising a reactor vessel (2) and a circulation loop. The circulation loop comprises at least one nozzle (6a) positioned for injection of reaction fluid from the circulation loop into the reactor vessel (2). In a second aspect the present invention relates to a method of mixing fluids in a bioreactor (1).

### FIELD OF INVENTION

The present invention relates to a bioreactor comprising a reactor vessel and a circulation loop. The circulation loop comprises a nozzle positioned at a wall of the reactor vessel for injection of reaction fluid from the circulation loop into the reactor vessel. In a second aspect the present invention relates to a method of mixing fluids in a bioreactor.

### BACKGROUND OF THE INVENTION

Stirred Tank Reactors (STR) with rotating agitators have been used for many years in fermentation and similar processes. In such reactors, mechanical agitators rotate within the reactor in order to mix the reaction fluids and thereby improve the degree of homogeneity. Whilst in many circumstances Stirred Tank Reactors can give adequate results, they may result in suboptimal mixing, often have a high power consumption and/or may exert high shear forces on the reactor contents.

One consequence of poor mixing is that gradients occur inside the bioreactor. Typically, cells consume dissolved oxygen within a few seconds, after which if the oxygen is not replenished, they begin to alter their metabolism, which drastically reduces the productivity.

There is often a trade-off between the extent of mixing provided in the bioreactor and the power consumption required to provide such mixing. Whilst conventional systems may be considered to be sufficient, there is a need for alternative or even improved systems which provide a good balance between mixing characteristics and power consumption.

There is also a trade-off between higher mixing and higher gas hold up. This means that as rotation rate is increased on an STR design both mixing and the gas hold up is increased even though this may be unwanted due to gradient formation due to hydrostatic pressure. It is a challenge within STR reactors that mixing time and oxygen/gas transfer cannot each be controlled independently.

Some alternative designs of bioreactor replace mechanical agitators with a pump to circulate liquid medium from the bioreactor through a gas entrainer and back to the reaction vessel. An example is disclosed in Principles of Fermentation Technology, 3^{rd} edition, Peter F. Stanbury, Allan Whitaker and Stephen J. Hall, which discloses a deep jet loop design with a nozzle placed centrally at the top of the reactor. The deep jet loop design is used in place of mechanical agitators.

A further alternative is disclosed in Fermentation and Biochemical Handbook, 3rd edition, Henry C. Vogel, and Celeste M. Torado, in which a submerged jet is discussed as a means providing air-agitated fermenters.

WO2019/030185 discloses a fermenter comprising one or more two phase injectors, the injectors providing oxygen for fermentation and circulating the fermentation broth. All injectors are provided in a lower part of the fermenter, within the fermenter, so that oxygen from the bubbles ejected from the injectors can be transferred to the liquid whilst the bubbles rise to the top of the liquid.

These conventional means of replacing a mechanical agitator of a Stirred Tank Reactor with an injector have been found to result in insufficient mixing within the bioreactor. There is therefore a need for a further alternative or improvement to conventional Stirred Tank Reactors and their alternatives which gives favourable mixing characteristics.

The present invention has been made from a consideration of this.

### SUMMARY OF THE INVENTION

Thus, according to a first aspect of the present invention, there is provided a bioreactor comprising a reactor vessel and a circulation loop,
the reactor vessel having a longitudinal axis defining an axial direction (a) and a radial direction (r) extending substantially perpendicular to said axial direction, the reactor vessel comprising at least one outlet providing fluid communication between the reactor vessel and the circulation loop, the reactor vessel comprising a vessel top, a vessel bottom, and a vessel wall,
the circulation loop comprising at least one pump, and at least nozzle positioned at the vessel wall for injection of reaction fluid from the circulation loop into the reactor vessel, there being a fluid pathway from the at least one outlet to the at least one nozzle via the at least one pump.

It has been found that mixing within the bioreactor can be achieved by passing reaction fluid through a circulation loop and redistributing the reaction fluid by injection back into the reactor vessel via at least one nozzle.

It will be understood that in the context of the present invention the reference to gas is any suitable gas, preferably a gas comprising oxygen, such as air. In some embodiments, the gas may be clean gasses or may be gas recirculated from the headspace of the reactor. The gas may be a mixture of multiple gases. Thus, the reaction fluid may be described as aerobic reaction fluid. In other words, the reaction fluid may be considered as oxygen-consuming reaction fluid.

It will be understood that a nozzle is a means for connection to a reactor vessel for injecting media into the vessel. A two phase nozzle is a nozzle which can contain two different medias, one in gas form, and the other in liquid form. The term nozzle is not intended to be limited to nozzles with circular cross sectional injection profiles, but additionally covers other cross sectional shapes, directional slits and the like. The nozzle facilitates introduction of fluid(s) into the reactor vessel from the recirculation loop.

In some embodiments, the circulation loop comprises 20 or fewer nozzles. Preferably the circulation loop comprises from 2 to 18 nozzles. Even more preferably the circulation loop comprises from 2 to 15 nozzles. Generally, the greater the number of nozzles, the shorter time taken to achieve mixing within the reactor vessel. However, a greater number of nozzles generally increases costs and operational complexity. The ranges outlined have been found to provide a particularly good balance between these factors, and particularly for the reactor sizes typically used in industry. The number of nozzles may be selected according to volume of the reactor vessel, with more nozzles generally being used for larger reactor vessels. The circulation loop may comprise 2-5 nozzles, 5-10 nozzles or 10-15 nozzles.

In some embodiments, at least one nozzle is positioned at a positive angle compared to a horizontal plane and at least one nozzle is positioned at a negative angle compared to a horizontal plane. It has been found that arranging the nozzles in this way further improves the mixing performance. This configuration of nozzles results in both upwards and downward angled jets, which further promotes mixing in the axial direction.

It will be understood that a positive angle is an angle above the horizontal plane. A negative angle is an angle below the horizontal plane.

In some embodiments, the angle to a horizontal plane for all nozzles is within the range −70° to +70°. Preferably the angle to a horizontal plane for all nozzles is within the range −60° to +60°. These angles are particularly beneficial as they further enhance mixing in the axial direction whilst also directing the injected jet away from the reactor wall and thereby maximising the mixing effect of the jet.

In some embodiments, at least one nozzle is positioned at a positive angle compared the radial direction and at least one nozzle is positioned at a negative angle compared to the radial direction. It has been found that arranging the nozzles in this way improves the mixing performance.

It will be understood that a positive angle is an angle to the right when viewed from the nozzle, facing the radial direction. Likewise, a negative angle is an angle to the left when viewed from the nozzle, facing the radial direction.

In some embodiments, the angle to the radial direction for all nozzles is within the range −50° to +50°. These angles are particularly beneficial as they further enhance mixing by directing the injected jet away from the reactor wall.

It will be understood that bioreactors are in general supplied with a nominal fill level. The nominal fill level of a reactor vessel is the level to which the reactor has been designed to be filled with liquid so that the reactor can operate with the appropriate headspace above the nominal fill level. Thus, the nominal fill level can be considered an intrinsic property of a reactor vessel of a bioreactor. Where the bottom of the reactor vessel is not flat, the height of the nominal fill level is measured from the lowest part of the reactor vessel. The position of the one or more nozzles can be considered in relation to the nominal fill level of the reactor vessel.

In some embodiments all nozzles are located below the nominal fill level. In some embodiments, all nozzles are located below half the height of the nominal fill level. Such configurations have been found to result in surprisingly good i.e. low mixing times. At least one of these nozzles may be a two phase nozzle. Preferably all of these nozzles are two phase nozzles. A combination of single phase and two phase nozzles may be used. The use of two phase nozzles enables the simultaneous introduction of gas, preferably oxygen-containing gas. Such configurations have been found to not only give good mixing characteristics, but also good transfer from gas bubbles to the reaction fluid.

In some embodiments, the circulation loop comprises at least two nozzles, at least one nozzle is located below half the height of the nominal fill level and at least one nozzle is located above half the height of the nominal fill level. Such configurations allow for mixing at different heights of the reaction fluid. Alternatively, all nozzles may be located below half the height of the nominal fill level.

In some embodiments, there are one or more nozzles positioned at a height of 25-50% of the height of the nominal fill level and/or there are one or more nozzles positioned at a height of 51-100% of the nominal fill level of the reactor vessel. Such configurations allow for simultaneous mixing at lower and upper parts of the reaction fluid.

Where one or more of the nozzles is a two phase nozzle, the mixing characteristics are also supplemented by favourable introduction of gas, aiding transfer of gas to the reaction fluid.

In some embodiments, all the nozzles are distributed on 2 or 3 horizontal planes. Preferably each horizontal plane has the same number of nozzles. Compared to apparatus in which all nozzles are arranged at a different height from the others, these configurations are generally easier to manufacture and are more cost effective. In these embodiments at least one nozzle in each plane may be a two phase nozzle.

This ensures that gas can be introduced at various heights throughout the reactor. Thus, in addition to favourable mixing, there is the further benefit that gas can be delivered into regions where it may otherwise become depleted.

It will be understood that the height of the reactor vessel is the distance from top to bottom of the reactor vessel in the axial direction. Where the bottom and/or the top is not flat, the height is the distance is from the lowest point of the reactor vessel to the top of the reactor wall, not including the top portion of the reactor.

In some embodiments, the circulation loop comprises a first set of nozzles, the first set of nozzles having one or more nozzles each of which is positioned within the range of 15-33% of the height of the reactor vessel, the circulation loop further comprising a second set of nozzles, the second set of nozzles having one or more nozzles each of which is positioned within the range of 40-75% of the height of the reactor vessel. The nozzles may be distributed on 2 horizontal planes. In alternative embodiments, the first set of nozzles may be within the range 10-39% of the height and the second set within the range 40-75% of the height. Alternatively, the first set may be within the range 5-25% of the height and the second set within the range 26-50% or 26-75% of the height.

In alternative embodiments, the circulation loop comprises a first set of nozzles, the first set of nozzles having one or more nozzles each of which is positioned within the range of 15-30% of the height of the reactor vessel, the circulation loop further comprising a second set of nozzles, the second set of nozzles having one or more nozzles each of which is positioned within the range of 30-55% of the height of the reactor vessel, the circulation loop further comprising a third set of nozzles, the third set of nozzles having one or more nozzles each of which is positioned within the range 60-75% of the height of the reactor vessel. The nozzles may be distributed on 3 horizontal planes.

In some embodiments, the circulation loop comprises a first set of nozzles, the first set of nozzles having one or more nozzles below 50% of the height of the reactor vessel, or below 40% of the height of the reactor vessel, or below 30% of the height of the reactor vessel. In some of the embodiments all the nozzles are positioned in this way i.e. there are no nozzles above 50% or 40% or 30% of the height of the reactor vessel respectively.

These configurations have been found to provide particularly good mixing characteristics.

In some embodiments, each nozzle that shares a common horizontal plane has an angle to the horizontal and/or angle to the radial direction that differs from the angle to the horizontal and/or angle to the radial direction respectively of every other nozzle in the common horizontal plane. In other words, for nozzles that share a horizontal plane, no two will share both a horizontal and radial angle. They may have the same radial angle but different horizontal angles, they may have the same horizontal angles but different radial angles or they may have different horizontal and radial angles. The result of this is that nozzles in the same plane provide jets in differing directions and therefore mixing in different directions.

Where nozzles share a common horizontal plane, preferably the angle of separation between all pairs of neighbouring nozzles in that plane is within the range: (360/number of nozzles in the plane)+/−10°. The nozzles can be considered to be distributed around the circumference of the reactor vessel, although the spacing between each pair of neighbouring nozzles need not be identical. This reduces the risk of having regions of low mixing.

In some embodiments, at least one nozzle is moveable to provide an adjustable angle of injection to the horizontal and/or angle to the radial direction. In some embodiments, all nozzles are moveable to provide an adjustable angle of injection to the horizontal and/or angle to the radial direction. When some or all of the nozzles are moveable, a bioreactor can be optimised for different conditions, for example different contents, reagents, reaction temperatures and viscosities. The use of moveable nozzles allows angles to be optimised, either during a reaction or in reactor downtime to account for different reactor conditions. For example, during a fed batch production the requirements can change leading to different optimal nozzle angles. This can be catered for by changing the angles to ensure constant optimised angle positions.

In some embodiments, the circulation loop further comprises cooling apparatus for cooling fluid from the reactor vessel. Preferably, the cooling apparatus is used as an alternative to cooling coils and/or a cooling jacket, which is the conventional cooling means used with Stirred Tank Reactors. Thus, in some embodiments where the circulation loop comprises cooling apparatus for cooling fluid from the reactor vessel, the reactor vessel does not comprise a cooling jacket and/or cooling coils. In alternative embodiments the cooling apparatus may be used in addition to cooling coils and/or a cooling jacket, to reduce the cooling burden on the cooling coils and/or the cooling jacket.

Conventional Stirred Tank Reactors typically control temperature using part of the surface as a cooling/heating surface. Area/Volume ratio decreases with greater size, so the cooling surface becomes insufficient for larger reactors. To combat this, extra surfaces, typically in the form of cooling coils, are dipped into the product, but these are difficult to clean and generate additional zones of gradients in the reactor. The provision of cooling apparatus in the circulation loop addresses these problems, as cooling can be achieved separate from the reactor vessel, therefore reducing the need for or obviating the need for a cooling jacket and/or cooling coils.

Also, by providing the alternative cooling means as described, the amount of material, typically steel, required is significantly reduced. Calculations show that a system in which the cooling is carried out outside of the reactor vessel, as part of the circulation loop, will typically require around 40% less steel than a conventional reactor with cooling coils and a cooling jacket. This presents a significant improvement since the reduced material requirements give rise to a system which is more sustainable as it requires less steel, it uses less energy to sterilize and is cheaper to manufacture. A reduction in the amount of material required by combining cooling means is also beneficial.

In some embodiments, the cooling apparatus comprises a gas inlet for supplying gas to the reaction fluid during cooling. In this way, productivity can be improved since required gasses, such as those comprising oxygen, can be added to the reaction fluid during the cooling procedure to ensure that the reaction fluid does not become depleted of the required gases during the cooling process, when the reaction fluid is not inside the reactor vessel.

In some embodiments, the reactor vessel is configured to operate with a headspace of from 10 to 50% of the volume of the reactor. Preferably all of the nozzles are located outside the headspace, below the nominal vessel fill level. In this way, when the reactor vessel is filled to a predetermined fill level, the nozzles will be below the fluid line and can have a greater influence on mixing compared with a nozzle positioned in the headspace. The headspace may be 10-40% of the volume of the reactor, or 10-30% of the volume of the reactor or 10-20% of the volume of the reactor, or 25-50% of the volume of the reactor.

It will be understood that headspace is a term of the art which refers to the volume above the liquid media inside a reactor vessel when the reactor vessel is considered to be full. The headspace arises because gravity makes heavier liquid collect in the bottom of the reactor vessel, leaving a headspace above the liquid.

Preferably, the reactor vessel does not comprise a mechanical agitator. In this way the recirculation system is provided as an alternative to conventional mechanical agitators as a means to provide mixing.

In some embodiments, at least one nozzle is a two phase nozzle. In other embodiments all of the nozzles are two phase nozzles. As a result, gas is injected to the reactor vessel at positions and/or multiple heights. Thus, mixing and gas introduction can be simultaneously optimised. One or more regulators may be used, meaning that the amount of gas delivered at different positions within the reactor vessel can be tailored according to the requirements of the reaction being conducted. Further, gas transfer can be controlled independently of mixing time.

In some embodiments the reactor vessel may further comprise a gas sparger. The gas sparger may be located at any suitable position, for example in the bottom half of the height of the nominal fill level or in the bottom third of the height of the reactor vessel. The gas sparger can facilitate the introduction of additional gas, preferably oxygen-containing gas.

According to a second aspect of the present invention, there is provided a method of mixing fluids in a bioreactor according to the first aspect of the invention, the method comprising the steps of:
withdrawing liquid through the at least one outlet of the reactor vessel,
circulating liquid through the circulation loop to the nozzle(s),
injecting the liquid through the nozzle(s) into the reactor vessel.

In this way, the mixing characteristics may be the same or even better than for a conventional Stirred Tank Reactor.

In some embodiments, the loop flow ratio is at least 5 times per hour. It will be understood that the term loop flow ratio defines the number of times per hour that the entire volume of the reactor vessel is passed through the recirculation system. The higher the loop flow ratio, the more times the reactor vessel contents are recirculated. A higher loop flow ratio generally results in improved mixing, but comes at an additional cost in terms of the equipment needed to facilitate the higher liquid transfer rate. In other embodiments, the loop flow ratio may be at least 10 times per hour, or at least 15 times per hour.

In some embodiments each nozzle has a nozzle speed in the range of 5 to 50 m/s, preferably in the range of 10 to 35 m/s. It will be understood that the term nozzle speed refers to the speed at which fluid(s) from the nozzle is travelling at the point at which it leaves the nozzle and enters the reaction vessel.

In some embodiments, the method further comprises the step of cooling reaction medium as it passes from the reactor vessel outlet to the nozzles. In this way, sufficient cooling can be obtained outside the reactor vessel which reduces or obvi-ates the need for cooling coils within the reactor vessel and/or the need for a cooling jacket. This can be advantageous for a number of reasons, including reduced risk for contamination by removing the need for cooling coils, and also greater scope to ret-rofit the existing systems. Also, as discussed above, by providing an alternative cooling means as described, the amount of material, typically steel, required is significantly reduced. Calculations show that a system in which the cooling is carried out outside of the reactor vessel, between the reactor vessel outlet and the nozzles, will typically require around 40% less steel than a conventional reactor with cooling coils and a cooling jacket. This presents a significant improvement since the reduced material requirements give rise to a system which is more sustainable as it requires less steel, it uses less energy to sterilize and is cheaper to manufacture.

Gas, preferably oxygen containing gas such as air can be added to the reaction fluid as it is cooled.

### BRIEF DESCRIPTION OF DRAWINGS

In the following description, embodiments of the invention will be described with reference to the schematic drawings in which:

FIG. 1 is a schematic representation of a bioreactor according to the invention;

FIG. 2 is a schematic representation showing one possible nozzle configuration for a bioreactor with 8 nozzles;

FIG. 3 is a further representation of the reactor shown in FIG. 2 in which the jet directions for the nozzles are shown;

FIG. 4a is a schematic representation of bioreactor with a circulation loop comprising cooling apparatus;

FIG. 4b is an expanded view showing the cooling apparatus shown in FIG. 4a;

FIG. 4c is a more advanced representation of the simplified view of the cooling apparatus shown in FIG. 4b;

FIG. 4d shows an alternative execution of the cooling system shown in FIG. 4b and FIG. 4c;

FIG. 5 is a graphic representation of STR-A and STR-B used in the simulations and referenced below;

FIG. 6 is a schematic representation of an adjustable nozzle, showing two possible nozzle angles;

FIG. 7 shows in graph form local maximum and average broth temperatures for a series of 2000 I reactors modelled;

FIG. 8 shows in graph form maximum and average broth temperatures for a series of 500 m³ reactors modelled;

FIG. 9 shows in graph form strain rates for the reactors referred to in connection with FIG. 8;

FIG. 10 a-d show schematic representations of the nozzle height configurations discussed in the ‘nozzle heights’ section below.

### DESCRIPTION OF EMBODIMENTS

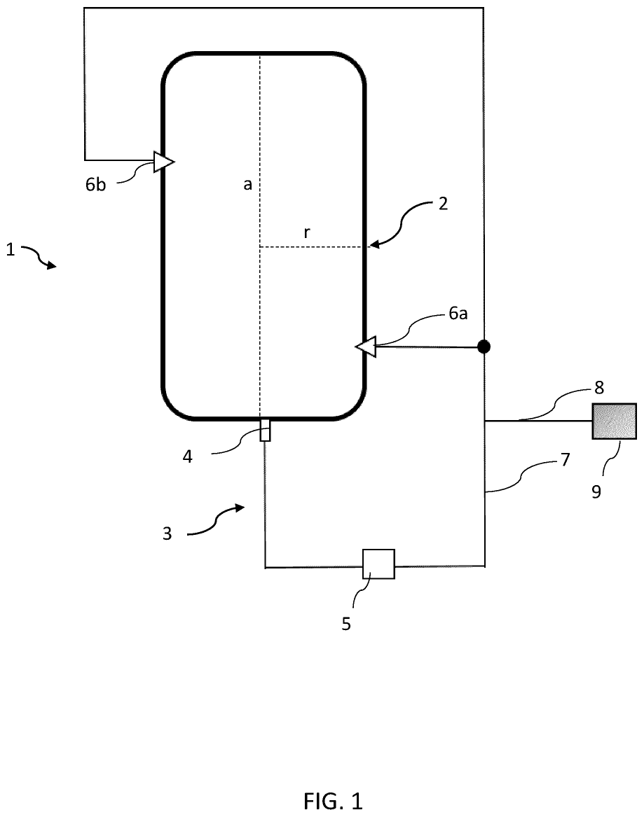

Referring now to the figures, a bioreactor generally designated 1 is shown in FIG. 1. The bioreactor 1 comprises a reactor vessel 2, which has a longitudinal axis defining an axial direction (a) and a radial direction (r) extending substantially perpendicular to said axial direction. It will be understood that the volume of the reactor vessel may be selected according to the application. In some embodiments the reactor vessel has a volume of as little as 10 m³ or less. However, for production units it is generally preferable to have a reactor vessel with a much larger volume. Thus, in alternative embodiments, the reactor vessel has a volume of at least 100 m³, for example in the range of 100 to 1000 m³, or in the range 200 to 600 m³. Reactor vessels of this size for bioreactors are well known.

The reactor vessel comprises at least one outlet 4 providing fluid communication between the reactor vessel 2 and circulation loop 3. In the embodiment shown the outlet is located at the bottom of the reactor vessel 2. In alternative embodiments, the outlet may be positioned on a side wall of the reactor vessel, preferably in the bottom third of the height of the reactor vessel. The diameter of the outlet can be selected according to the application and the reactor size, with a larger diameter outlet generally being preferable for a larger volume reactor vessel. The outlet may preferably have a diameter of 600 mm to conform with standardised valve sizes. Whilst one outlet 4 is shown in FIG. 1, the reactor vessel 2 may alternatively comprise more outlets providing fluid communication between the reactor vessel 2 and circulation loop 3, for example two to four outlets.

The circulation loop 3 comprises a pump 5, a gas inlet 8 connected to a gas supply 9 and two nozzles 6a, 6b positioned for injection of reaction fluid from the circulation loop into the reactor vessel. For the sake of simplicity, a single pump 5 is shown, but it will be understood that in other embodiments multiple pumps may be used. In the embodiment shown, the lower nozzle is a two phase nozzle. The gas inlet 8 is positioned to feed the two-phase nozzle from the gas source. The gas inlet may be connected directly to the, or where appropriate each, of the two phase nozzles. For these embodiments, two phase nozzles will comprise a mixing unit in which gas and liquid are mixed prior to injection and a directing nozzle which has the desired height and angles in relation to the reactor vessel, the directing nozzle being the part Which injects the mixture into the reactor vessel accordingly.

In some embodiments, the supply of gas to each nozzle can be individually regulated. In some embodiments, the flow of reaction fluid to each nozzle can be individually regulated. Preferably, the flow of gas and reaction fluid to each nozzle is individually regulated such that the amount of gas and reaction fluid delivered by each nozzle is tailored according to the reaction requirements. It may be, for example, that nozzles located higher within the reactor vessel deliver less gas/oxygen than those lower down, or even no gas/oxygen. Where not all nozzles are two phase the gas inlet can be located accordingly.

All of the nozzles may be two phase nozzles. In this way, gas can be delivered at multiple positions and/or heights throughout the reactor vessel.

There is a fluid pathway 7 from the at least one outlet 4 to each of the nozzles 6a, 6b via the pump 5. The fluid pathway may comprise suitable connection conduits as well known to the skilled person.

In the embodiment shown, the first nozzle is positioned at 20% of the height of the reactor vessel and the second nozzle is at 55% of the height of the reactor vessel. The first nozzle has an angle to the horizontal of 20° and an angle to the radial direction of 30°. The second nozzle has an angle to the horizontal of −30° and an angle to the radial direction of 15°. The nozzles are positioned on diametrically opposed sides of the reactor vessel.

Whilst the embodiment of FIG. 1 shows a two nozzle configuration, other configurations are also possible. In the simplest form, a single nozzle could be used. Likewise, multiple nozzles either at the same height or positioned at different heights could be used.

It will be understood that any suitable nozzle(s) may be used. An example of a two-phase nozzle that can be used according to the present invention is the GEA Venturi Saturator.

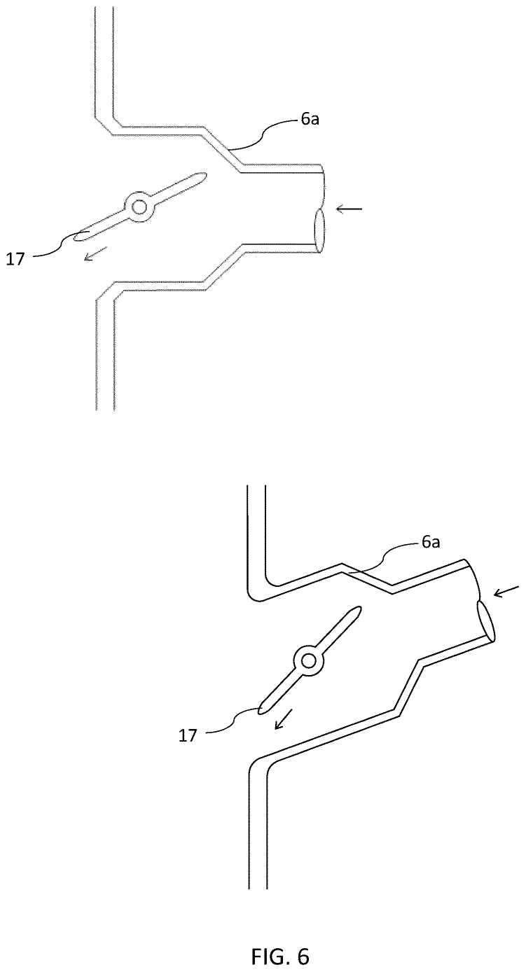

For nozzles that are moveable i.e. the injection angles achieved by the nozzle can be altered, an adjustable plate can be used, preferably at the centre of the nozzle, which can deflect the jet accordingly from the angle at which the nozzle is mounted to the wall of the reaction vessel. Thus, in some embodiments one or more nozzles, preferably all nozzles, may comprise a deflector plate 17. This can be seen in FIG. 6.

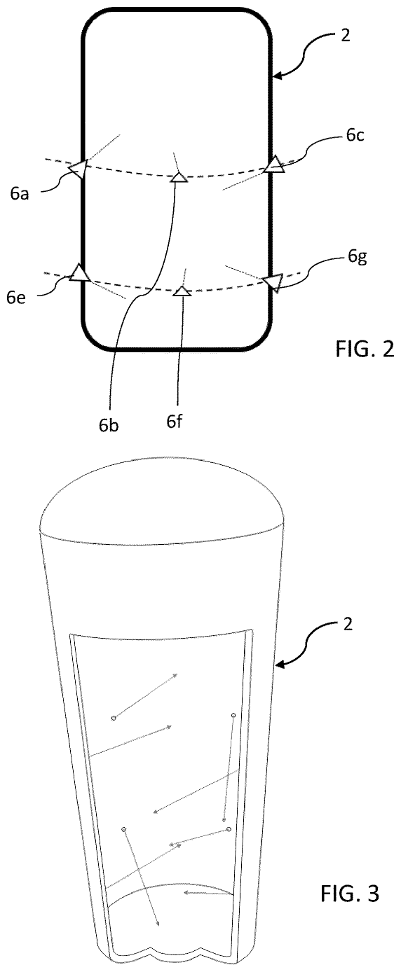

Referring to FIG. 2, a bioreactor with 8 nozzles is shown. The nozzles are distributed over two horizontal planes, each with 4 nozzles. The first horizontal plane is at 30% of the height of the reactor vessel and the second horizontal plane is at 60% of the height of the reactor vessel. For the nozzles that share a horizontal plane, the angle between each pair of neighbouring nozzles is 90°. Nozzles 6d and 6h are not shown in the figure.

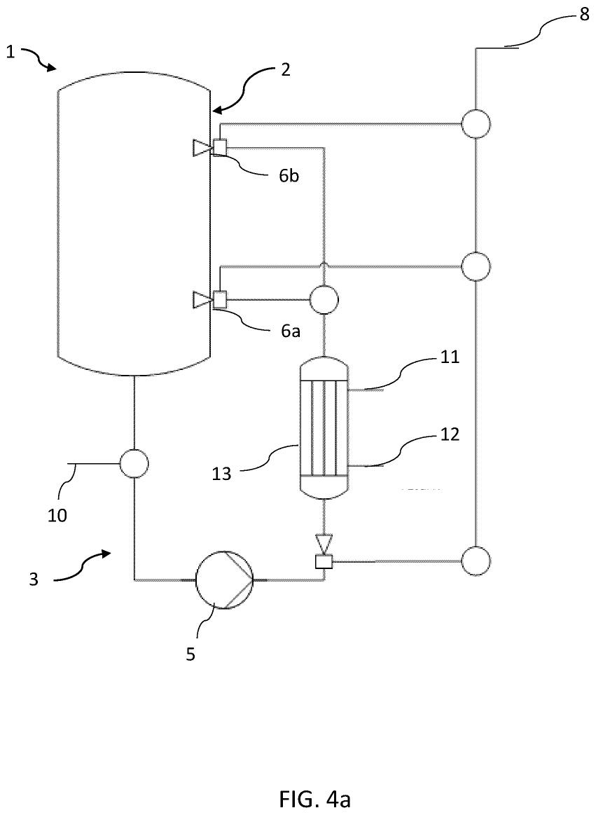

FIG. 3 shows the resulting injection jets from the configuration shown in FIG. 2. Preferably the nozzles are arranged so that the paths of the injected jets do not cross.

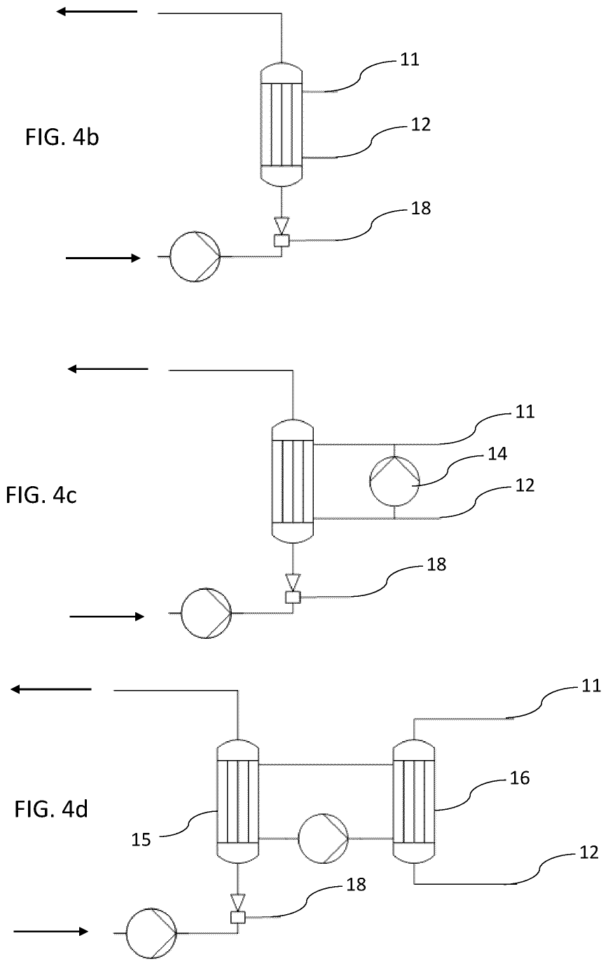

FIG. 4a shows an embodiment of the bioreactor 1 in which the circulation loop 3 further comprises cooling apparatus 13 for cooling reaction fluid from the reactor vessel 2. The cooling apparatus 13 further comprises chilled water supply 11 and chilled water return 12.

An expanded view of the section with cooling apparatus 13 can be seen in FIG. 4b. A more detailed view is shown in FIG. 4c where a pump of the cooling apparatus 14 is also shown. FIG. 4d shows an alternative execution in which the cooling apparatus comprises two chambers 15, 16.

In the embodiments shown there is a cooling apparatus gas inlet 18 which can be used to supply gas to the reaction fluid during cooling. In this way productivity can be improved by preventing or reducing the extent of depletion of gas during cooling when the reaction fluid is outside of the reaction vessel.

A number of tests were conducted to investigate the performance of embodiments of the inventions. The tests and corresponding results are described below.

### Simulations

### Baseline Performance STR—Virtual Test Bench

A virtual test bench was implemented to establish stirred tank reactor performance in order to provide a comparison for subsequent modelling to test bioreactor configurations in which the mechanical agitators are replaced by a fluid circulation system.

The tests were conducted on GEA proprietary custom-made software, but similar tests could be conducted using commercially available software such as ANSYS Fluent, M-star or Siemens CCM+.

Two stirred tank reactors were modelled initially, as follows:

STR reactor geometry

| Parameter | STR-A | STR-B |
|---|---|---|
| Reactor diameter D_{cyl} (mm) | 674 | 1100 |
| Reactor height (m) | 1.45 | 2.2 |
| Reactor volume (l) | 500 | 2000 |
| Agitator tip-to-tip diameter Dₜ (mm) | 225 | 367 |
| Dₜ/D_{cyl} | 0.33 | 0.33 |

The basic process conditions used in the modelling were as follows:

| | | STR-A | STR-B |
|---|---|---|---|
| Broth viscosity description | | Newtonian | Newtonian |
| Broth density, ρ | kg/m³ | 1200 | 1200 |
| Broth viscosity, μ | cP | 200 | 200 |
| Broth heat capacity, C | J/kg-K | 4182 | 4182 |
| Broth heat conductivity, k | W/m-K | 0.6 | 0.6 |
| Uniform metabolistic heat | kW/m³ | 15 | 15 |
| generation, qₘ | | | |
| Cooling surface temperature, T_{c} | ° C. | 12.5 | 12.5 |
| Agitator speed, RPM | 1/min | 540 | 333 |
| Agitator tip speed, Uₜ | m/s | 6.4 | 6.4 |

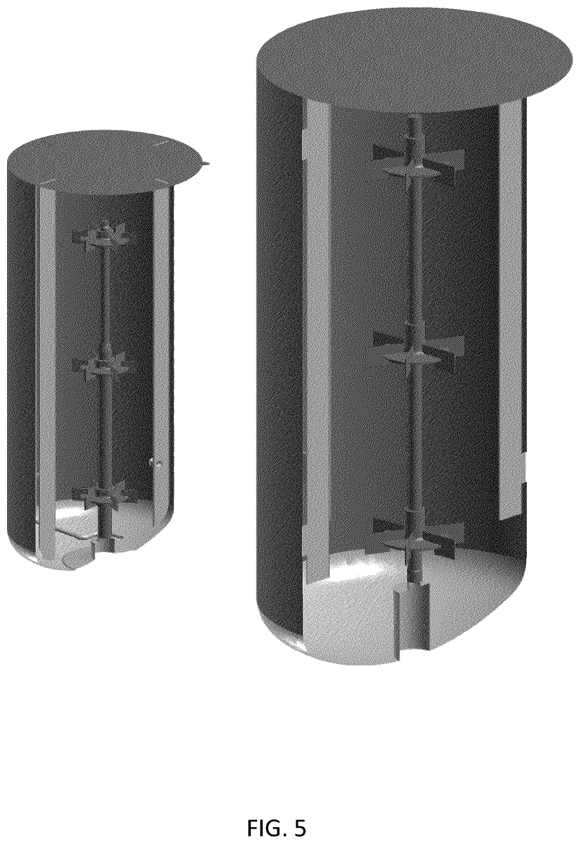

Graphical representations of the two reactors can be seen in FIG. 5.

Simulations were conducted to establish the mixing times for these reactors. Mixing times were determined as follows. First, the virtual test was allowed to run for enough time to establish a quasi-steady flow field. Next a blob of ink was inserted just below the filling level to mimic food or substrate injection. The spread of the ink in the bioreactor was then tracked. The virtual test was stopped when the concen-tration of ink was uniform [i.e. (max-min)/avg=0.005]. The mixing time was then determined by calculating the duration of time from ink insertion time to virtual test stop time.

The simulations gave a mixing time for STR-A of 42.2 seconds and a mixing time for STR-B of 43.6 seconds. It can therefore be concluded that the mixing time for both reactors was essentially unchanged regardless of the difference in reactor volume for STR-A and STR-B when the agitator speed remained the same in both.

The same conditions were then used to model the mixing time of different nozzle configurations, without the rotating agitators.

In all tests the vessel fill level was equivalent to the reactor height. In other words, the virtual tests were conducted with the top surface at nominal filling level, excluding the headspace found in a physical reactor.

### JLR_B—8 Nozzles

Reactor conditions

| | |
|---|---|
| Reactor volume: | 2000 l |
| Nozzle speed: | 40 m/s |
| Loop flow ratio: | 35 times per hour |

Nozzle positions and angles

| | | | Direction | Direction |
|---|---|---|---|---|
| Nozzle | Nozzle | Angular wall | compared to | compared to |
| number | height | position | horizontal | radial |
| 1 | 0% | 0 | 90 | 0 |
| | (bottom) | (Rnoz = | | |
| | | 50% Rwall) | | |
| 2 | 0% | 180 | 90 | 0 |
| | (bottom) | (Rnoz = | | |
| | | 50% Rwall) | | |
| 3 | 20% | 60 | 0 | 0 |
| 4 | 20% | 180 | 0 | 0 |
| 5 | 20% | 300 | 0 | 0 |
| 6 | 65% | 60 | 0 | 0 |
| 7 | 65% | 180 | 0 | 0 |
| 8 | 65% | 300 | 0 | 0 |

Overall mixing time: 45 s

### JLR_F—8 Nozzles

Reactor conditions

| | |
|---|---|
| Reactor volume: | 2000 l |
| Nozzle speed: | 58 m/s |
| Loop flow ratio: | 35 times per hour |

Nozzle positions and angles

| | | | Direction | Direction |
|---|---|---|---|---|
| Nozzle | Nozzle | Angular wall | compared to | compared to |
| number | height | position | horizontal | radial |
| 1 | 0% (bottom) | 0 (R = 50% Rwall) | 90 | 0 |
| 2 | 0% (bottom) | 180 | 90 | 0 |
| | | (R = 50% Rwall) | | |
| 3 | 20% | 60 | 0 | 0 |
| 4 | 20% | 180 | 0 | 0 |
| 5 | 20% | 300 | 0 | 0 |
| 6 | 65% | 60 | 0 | 0 |
| 7 | 65% | 180 | 0 | 0 |
| 8 | 65% | 300 | 0 | 0 |

Overall mixing time: 34 s

### JLR_K—8 Nozzles

Reactor conditions

| | |
|---|---|
| Reactor volume: | 2000 l |
| Nozzle speed: | 40 m/s |
| Loop flow ratio: | 35 times per hour |

Nozzle positions and angles

| | | | Direction | Direction |
|---|---|---|---|---|
| Nozzle | Nozzle | Angular wall | compared to | compared to |
| number | height | position | horizontal | radial |
| 1 | 5% | 0° | 10° | −20° |
| 2 | 5% | 90° | 20° | −10° |
| 3 | 5% | 180° | 15° | −30° |
| 4 | 5% | 270° | 0° | 0° |
| 5 | 45% | 0° | −35° | 0° |
| 6 | 45% | 90° | −60° | 25° |
| 7 | 45% | 180° | 30° | 0° |
| 8 | 45% | 270° | 0° | 10° |

Overall mixing time: 40 s

It can be seen that each of JLR_B, JLR_F and JLR_K provides a mixing time that is similar to or less than the equivalent stirred tank reactor.

### Lower Nozzles Only (JLR D and E)

Reactor volume: 2000 I
JLR_D: 8 bottom vertical nozzles
Nozzle speed: 58 m/s
Loop flow ratio: 16 times per hour
Overall mixing time: >100 s
JLR_E: 8 bottom nozzles, each 10° from vertical/axial direction
Nozzle speed: 58 m/s
Loop flow ratio: 16 times per hour
Overall mixing time: >100 s

Thus, it can be seen that tested configurations with the same number of nozzles, but placement only at the bottom of the reactor resulted in significantly higher mixing times in these particular simulations, over twice that of the STR baseline and the other nozzle configurations tested above, namely JLR_B, JLR_F and JLR_K.

It has also been hypothesised that having nozzles only in the lower part of the reactor vessel could result in oxygen deficiencies in the upper part of the reaction fluid as oxygen gets used up before gas bubbles reach the top. In some circumstances it may be beneficial to introduce gases at an appropriate position to account for this.

### Comparison of Power Consumption

| | STR-B | JLR_B | JLR_F | JLR_K |
|---|---|---|---|---|
| Agitator power (kW/m3) | ^{~}9 | — | — | — |
| Nozzle power (kW/m3) | — | 9.25 | 19.5 | 9.25 |

It can therefore be seen the JLR_B and JLR_F had a power consumption comparable to the stirred tank reactor modelled. JLR_F had a higher power consumption, by virtue of the higher nozzle speed compared to the other examples. However, it also provided a reduced mixing time.

### Comparison of Broth Temperature

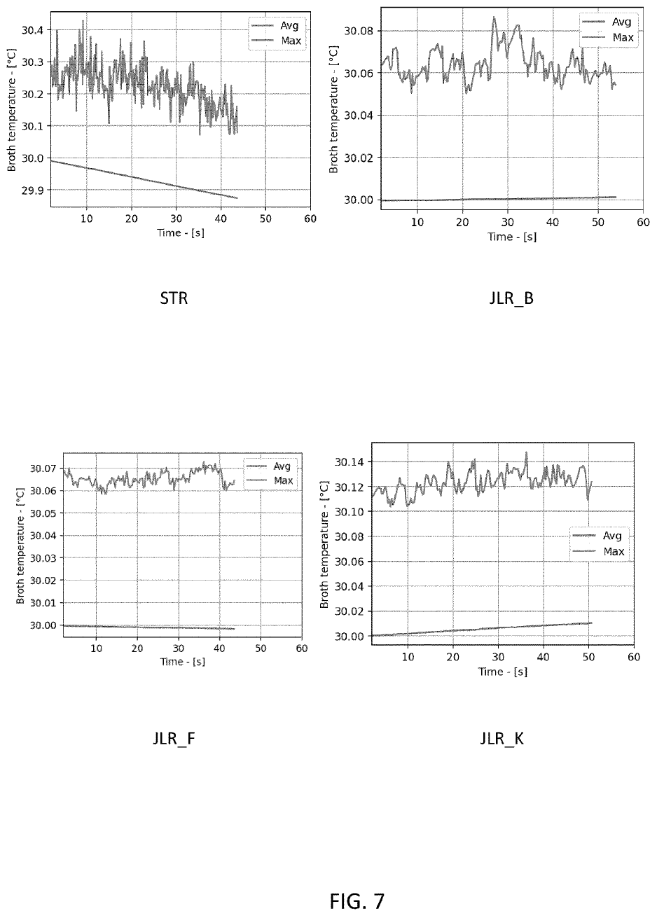

Broth temperature results can be seen in FIG. 7. It can be seen that for the Stirred Tank Reactor the average and maximum values are closely aligned. The same is true for JLR_B, JLR_F and JLR_K. This reflects good mixing. If the mixing had been inadequate there would have been an increasing average temperature over time. The relationship shown between average temperature and maximum temperature indi-cates that the observed extent of mixing is acceptable.

### Larger Reactor Vessel—Baseline STR Performance

Two more stirred tank reactors with larger volumes than the first two were also modelled, as follows:

| Parameter | STR-C | STR-D |
|---|---|---|
| Reactor diameter D_{cyl} (m) | 5 | 6.8 |
| Reactor height (m) | 10.525 | 14.229 |
| Reactor volume (m³) | 200 | 500 |
| Agitator tip-to-tip diameter Dₜ (m) | 5.0 | 6.8 |
| Dₜ/D_{cyl} | 0.33 | 0.33 |

| | | 200 m³ | 500 m³ |
|---|---|---|---|
| Broth viscosity description | | Newtonian | Newtonian |
| Broth density, ρ | kg/m³ | 1200 | 1200 |
| Broth viscosity, μ | cP | 200 | 200 |
| Broth heat capacity, C | J/kg-K | 4182 | 4182 |
| Broth heat conductivity, k | W/m-K | 0.6 | 0.6 |
| Uniform metabolistic heat | kW/m³ | 15 | 15 |
| generation, qₘ | | | |
| Cooling surface temperature, T_{c} | ° C. | 20 | 20 |
| Agitator speed, RPM | 1/min | 73 | 54 |
| Agitator tip speed, Uₜ | m/s | 6.4 | 6.4 |

These were found to give mixing times as follows:
200 m³-185 s
500 m³-232 s
   The larger vessels are intended to reflect the volumes that would typically be found in industrial processes.
   The same conditions were then used to model the mixing time of two further nozzle configurations, without rotating agitators.

### JLR L—12 Nozzles

Reactor volume: 500 m³

| | | | Direction | Direction |
|---|---|---|---|---|
| Nozzle | Nozzle | Angular wall | compared to | compared to |
| number | height | position | horizontal | radial |
| 1 | 25% | 0° | 25° | 10° |
| 2 | 25% | 90° | 0° | −5° |
| 3 | 25% | 180° | 20° | 0° |
| 4 | 25% | 270° | −5° | −30° |
| 5 | 50% | 0° | −20° | −5° |
| 6 | 50% | 90° | 15° | 40° |
| 7 | 50% | 180° | −10° | −20° |
| 8 | 50% | 270° | 30° | 45° |
| 9 | 75% | 0° | −10° | 20° |
| 10 | 75% | 90° | 30° | −15° |
| 11 | 75% | 180° | −15° | 10° |
| 12 | 75% | 270° | 20° | −15° |

Nozzle speed: 20 m/s
Loop flow ratio: 35 times per hour
Overall mixing time: 164 s

### JLR_N

Reactor volume: 500 m³

| | | | Direction | Direction |
|---|---|---|---|---|
| Nozzle | Nozzle | Angular wall | compared to | compared to |
| number | height | position | horizontal | radial |
| 1 | 25% | 0° | −30° | 25° |
| 2 | 25% | 75° | 60° | 0° |
| 3 | 25% | 145° | −30° | −10° |
| 4 | 25% | 215° | 60° | 30° |
| 5 | 25% | 290° | −30° | −20° |
| 6 | 60% | 0° | 60° | 15° |
| 7 | 60% | 75° | −60° | 20° |
| 8 | 60% | 145° | 0° | −40° |
| 9 | 60% | 215° | 60° | −10° |
| 10 | 60% | 290° | −60° | 25° |
| 11 | 90% | 0° | −60° | −15° |
| 12 | 90% | 75° | 10° | −35° |
| 13 | 90% | 145° | −60° | 0° |
| 14 | 90% | 215° | 10 | −30° |
| 15 | 90% | 290° | −60° | −40° |

Nozzle speed: 20 m/s
Loop flow ratio: 35 times per hour
Overall mixing time: 121 s
   It can be seen that each of JLR_L and JLR_N provides a mixing time that is less than the equivalent stirred tank reactor.

### Comparison of Power Consumption

| | Power - | |
|---|---|---|
| | kW/m3 | Comments |
| 500 m3 STR | 1.75 | Simulated STR |
| 500 m3 JLR_L | 2.3 | Ideal pumping power based on flow rate |
| | | and dynamic head in nozzle |
| 500 m3 JLR_N | 2.3 | Ideal pumping power based on flow rate |
| | | and dynamic head in nozzle |

Thus, the JLR_L and JLR_N tests give power consumption per unit volume that is comparable to the equivalent STR reactor.

### Comparison of Broth Temperature

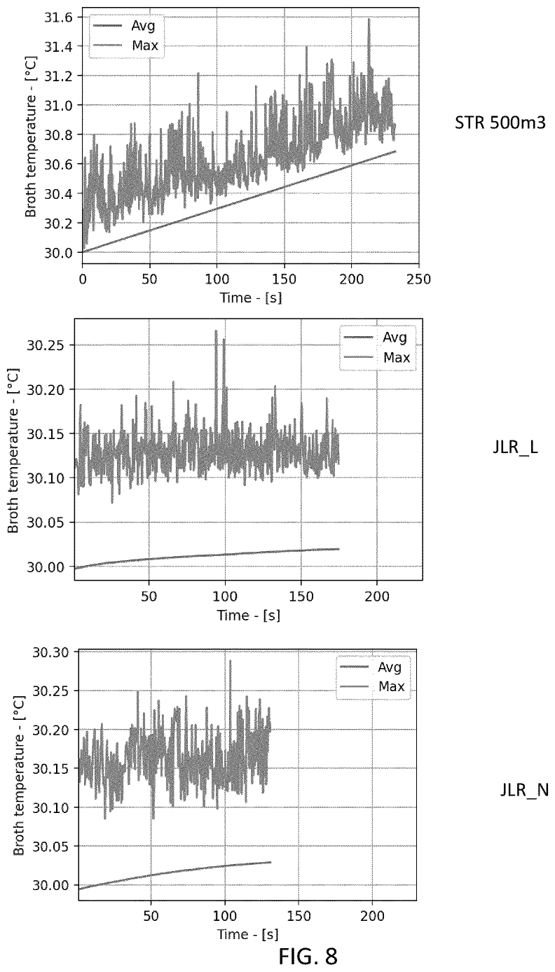

The broth temperature results for the larger reactors can be seen in FIG. 8. It can be seen that for the Stirred Tank Reactor the average and maximum values in-crease with time. This is seen to a much lesser extent with JLR_L and JLR_N, which essentially level out. The difference between the average and maximum temperatures is much smaller for JLR_L and JLR_N than for the comparable stirred tank reactor. This shows a better temperature uniformity in the JLR_L and JLR_N examples than in the comparable stirred tank reactor. Bioreactor contents are often very sensitive to temperature, and so the ability to maintain the temperature within a narrow range is beneficial for factors such as reaction rate and output.

### Comparison of Strain Rate

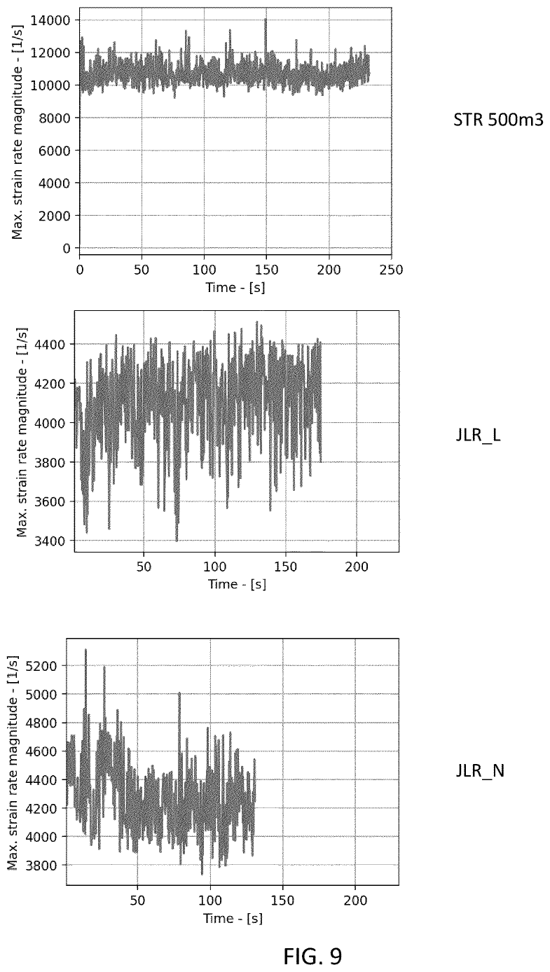

The strain rates can be seen in FIG. 9. It can be seen that the strain rates for JLR_L and JLR_N are comparable to each other, whereas for the comparable STR reactor the strain rate is approximately twice as much. It can be concluded that the circulation loop method of mixing has the potential to provide a lower risk of cell damage than the use of a mechanical agitator. This can be beneficial for reactions using cells which are particularly sensitive to strain and therefore prone to damage.

In summary, it can therefore be seen that the present invention provides apparatus and methods which give results which are at least comparable to the equivalent conventional stirred tank reactor setup, and in many respects as demonstrated above give improved results according to numerous parameters.

### Pilot Reactor Testing

Following the simulation testing, tests were conducted on a 2000 litre pilot reactor to establish mixing times and gas transfer efficiencies from bubbles to the liquid phase (referred to as kLa tests). The tests were conducted based on an 8-nozzle configuration in which nozzles could independently be activated, deactivated and se-lectively operated as single or two-phase nozzles. The 8 nozzles were divided over two planes and the 4 nozzles in each plane were evenly distributed around the diameter of the reactor vessel.

The pilot tests confirmed that the present invention can provide a feasible alternative to a stirred tank reactor by provision of one or more nozzles in the circulation loop to give suitable mixing time, and where appropriate, suitable gas transfer rates.

### Nozzle Heights

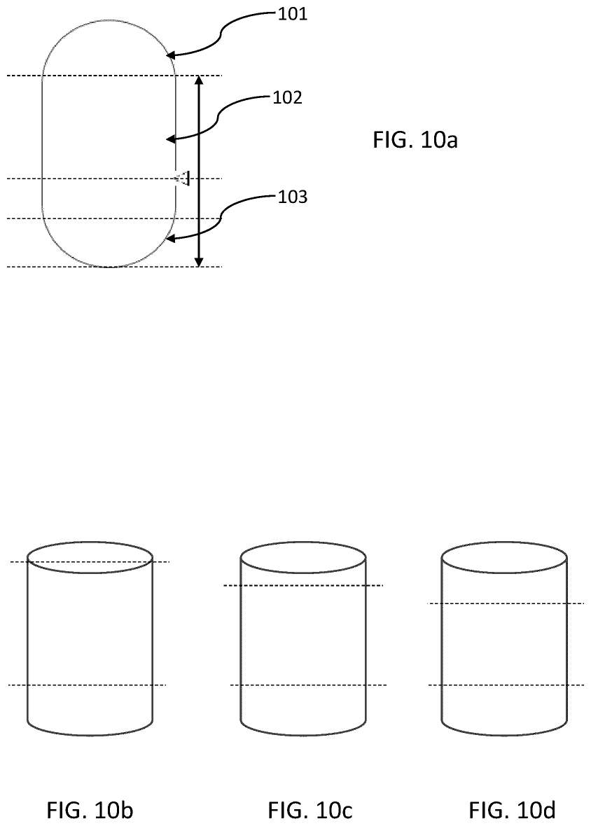

By way of an illustrative example, consider a reactor vessel with a volume of 100 m³. For simplicity, the reactor is modelled as a cylinder with a flat top and a flat bottom. The reactor has a height of 8 m and a radius of 2 m. The circulation loop comprises a single nozzle that is positioned at a height of 2 m from the bottom of the reactor. Thus, the nozzle can be considered to be located at 25% of the height of the reactor. This is shown in FIG. 10b, where the lower line represents the nozzle height and the upper line represents the reactor height.

The position of the nozzle can also be defined in terms of the nominal fill level of the reactor. In these circumstances the intended purpose of the reactor is also a consideration since the volume of the headspace needs to be accounted for.

If the reactor is intended to operate with a 10% headspace i.e. a headspace volume of 10 m³, a reactor with the same dimensions as discussed above would have a nominal fill level positioned at 7.1 m from the bottom of the reactor. Thus, the nozzle at 2 m from the base of the reactor would be considered to be at 28% of the height of the nominal fill level. This is shown in FIG. 10c where the lower line represents the nozzle height (same as FIG. 10b) and the upper line represents the nominal fill level of this reactor.

If a reactor of the same dimensions is created to operate with a 30% headspace i.e. a headspace of 30 m³, the nominal fill level would be positioned at around 5.6 m from the bottom of the reactor. Thus, the nozzle at 2 m from the base of the reactor would be considered to be at 36% of the height of the nominal fill level. This is shown in FIG. 10c where the lower line represents the nozzle height (same as in FIG. 10b & c) and the upper line represents the nominal fill level of this reactor.

It will therefore be understood that the nozzle position may be defined either in terms of the total height of the reactor vessel or alternatively may be defined in terms of the nominal fill level, accounting for the intended headspace.

For further comparison, a further reactor can be considered, also having a volume of 100 m³, but with a height of 2 m and a radius of 4 m. A nozzle positioned at 20% of the height of reactor would therefore be 40 cm from the bottom of the reactor.

In practice, reactor vessels may not have a flat bottom and/or a flat top. In such circumstances, the height of the reactor vessel is intended to be the height from the lowest part of the reactor vessel to the uppermost part of the reactor vessel wall, not including the height of any none-flat top part. This is shown by the double-headed arrow in FIG. 10a.

### Itemized List of Enumerated Embodiments

EE1. A bioreactor (1) comprising a reactor vessel (2) and a circulation loop (3),
the reactor vessel (2) having a longitudinal axis defining an axial direction (a) and a radial direction (r) extending substantially perpendicular to said axial direction, the reactor vessel comprising at least one outlet (4) providing fluid communication between the reactor vessel (2) and the circulation loop (3),
the circulation loop comprising at least one pump (5), a gas inlet (8) connected to an gas supply (9) and at least two nozzles (6a, 6b) positioned for injection of reaction fluid from the circulation loop into the reactor vessel (2), at least one of the nozzles being a two-phase nozzle, there being a fluid pathway (7) from the at least one outlet (4) to each of the nozzles (6a, 6b) via the at least one pump (5),
characterised in that a first nozzle (6a) is positioned in a bottom third of the height of the reactor vessel and a second nozzle (6b) is positioned in a top two thirds of the height of the reactor vessel.

EE2. A bioreactor according to EE1, wherein the circulation loop comprises 20 or fewer nozzles, preferably from 5 to 18, even more preferred from 8 to 15.

EE3. A bioreactor according to any one of EE1 or 2, wherein at least one nozzle is positioned at a positive angle to a horizontal plane and at least one nozzle is positioned at a negative angle to a horizontal plane.

EE4. A bioreactor according to any preceding EE, wherein the angle to a horizontal plane for all nozzles is within the range −70° to +70°, preferably the angle to a horizontal plane for all nozzles is within the range −60° to +60°.

EE5. A bioreactor according to any preceding EE, wherein at least one nozzle is positioned at a positive angle compared to the radial direction and at least one nozzle is positioned at a negative angle compared to the radial direction.

EE6. A bioreactor according to any preceding EE, wherein the angle to the radial direction for each nozzle is within the range −50° to +50°.

EE7. A bioreactor according to any preceding EE, wherein all the nozzles are distributed on 2 or 3 horizontal planes and at least one nozzle in each plane is a two phase nozzle, preferably each horizontal plane has the same number of nozzles.

EE8. A bioreactor according to EE7, wherein all nozzles are distributed on 2 horizontal planes, the first of which within the range of 15-33% of the height of the reactor vessel and the second of which is within the range of 50-75% of the height of the reactor vessel.

EE9. A bioreactor according to EE7, wherein all nozzles are distributed on 3 horizontal planes, the first of which within the range of 15-30% of the height of the reactor vessel, the second of which is within the range of 30-55% of the height of the reactor vessel and the third of which is within the range 60-75% of the height of the reactor vessel.

EE10. A bioreactor according to any one of EE7 to 9, wherein each nozzle that shares a common horizontal plane has an angle to the horizontal and/or angle to the radial direction that differs from the angle to the horizontal and/or angle to the radial direction respectively of every other nozzle in the common horizontal plane.

EE11. A bioreactor according to any one of EE7 to 10, wherein the angle between each pair of neighbouring nozzles in the same horizontal plane is within the range: (360/number of nozzles in the plane)+/−10°.

EE12. A bioreactor according to any preceding EE, wherein at least one nozzle is moveable to provide an adjustable angle of injection to the horizontal and/or angle to the radial direction, preferably all nozzles are moveable to provide an adjustable angle of injection to the horizontal and/or angle to the radial direction.

EE13. A bioreactor according to any preceding EE, wherein the circulation loop further comprises cooling apparatus for cooling reaction fluid from the reactor vessel.

EE14. A bioreactor according to EE 13, wherein the cooling apparatus comprises a gas inlet for supplying gas to the reaction fluid during cooling.

EE15. A bioreactor according to any preceding EE, wherein the reactor vessel is configured to operate with a headspace of up to 30% of the total volume of the reactor.

EE16. A bioreactor according to EE 15, wherein all of the nozzles are located outside of the headspace, below the reactor vessel fill level.

EE17. A bioreactor according to any preceding EE, wherein the reactor vessel does not comprise a mechanical agitator.

EE18. A bioreactor according to any preceding EE, wherein all the nozzles are two phase nozzles.

EE19. A method of mixing fluids in a bioreactor according to EE 1 to 18, the method comprising the steps of:
withdrawing liquid through the at least one outlet of the reactor vessel,
circulating liquid through the circulation loop to the nozzles,
injecting the liquid through the nozzles into the reactor vessel.

EE20. A method according to EE 19, wherein the loop flow ratio is at least 5 times per hour.

EE21. A method according to any one of EE 19 or 20, wherein each nozzle has a nozzle speed in the range of 5 to 50 m/s, preferably in the range of 10 to 35 m/s.

EE22. A method according to any one of EE 19 to 21, further comprising the step of cooling reaction fluid as it passes from the reactor vessel outlet to the nozzles.

### LIST OF REFERENCE NUMERALS

1 bioreactor
2 reactor vessel
3 circulation loop
4 outlet
5 pump
6a-6h nozzles
7 fluid pathway
8 gas inlet
9 gas supply
10 inlet for additional media
11 chilled water supply
12 chilled water return
13 cooling apparatus
14 pump of cooling apparatus
15 first chamber
16 second chamber
17 deflector plate
18 cooling apparatus gas inlet
101 vessel top
102 vessel wall
103 vessel bottom

What is claimed is:

1. A bioreactor comprising a reactor vessel and a circulation loop,
the reactor vessel having a longitudinal axis defining an axial direction and a radial direction extending substantially perpendicular to the axial direction, the reactor vessel comprising at least one outlet providing fluid communication between the reactor vessel and the circulation loop, the reactor vessel comprising a vessel top, a vessel bottom, and a vessel wall, and
the circulation loop comprising at least one pump, and at least one nozzle positioned at the vessel wall for injection of reaction fluid from the circulation loop into the reactor vessel, there being a fluid pathway from the at least one outlet to the at least one nozzle via the at least one pump.

2. A bioreactor according to claim 1, wherein the circulation loop comprises 20 or fewer nozzles.

3. A bioreactor according to claim 2, wherein at least one nozzle is positioned at a positive angle to a horizontal plane and at least one nozzle is positioned at a negative angle to a horizontal plane.

4. A bioreactor according to claim 1, wherein the angle to a horizontal plane for all nozzles is within the range −70° to +70°.

5. A bioreactor according to claim 1, wherein there are at least two nozzles and least one nozzle is positioned at a positive angle compared to the radial direction and at least one nozzle is positioned at a negative angle compared to the radial direction.

6. A bioreactor according to claim 1, wherein the angle to the radial direction for each nozzle is within the range −50° to +50°.

7. A bioreactor according to claim 1, wherein the reactor vessel has a nominal fill level.

8. A bioreactor according to claim 7, wherein at least one nozzle is a two phase nozzle.

9. A bioreactor according to claim 1, wherein the reactor vessel has a nominal fill level and there are at least two nozzles, wherein at least one nozzle is located below half the height of the nominal fill level and at least one nozzle is located above half the height of the nominal fill level and below the nominal fill level.

10. A bioreactor according to claim 9, wherein there are one or more nozzles positioned at a height of 25-50% of the height of the nominal fill level or wherein there are one or more nozzles positioned at a height of 51-100% of the nominal fill level of the reactor vessel.

11. A bioreactor according to claim 2, wherein the circulation loop comprises a first set of nozzles, the first set of nozzles having one or more nozzles each of which is positioned within the range of 15-33% of the height of the reactor vessel, the circulation loop further comprising a second set of nozzles, the second set of nozzles having one or more nozzles each of which is positioned within the range of 40-75% of the height of the reactor vessel.

12. A bioreactor according to claim 2, wherein the circulation loop comprises a first set of nozzles, the first set of nozzles having one or more nozzles each of which is positioned within the range of 15-30% of the height of the reactor vessel the circulation loop further comprising a second set of nozzles, the second set of nozzles having one or more nozzles each of which is positioned within the range of 30-55% of the height of the reactor vessel, the circulation loop further comprising a third set of nozzles, the third set of nozzles having one or more nozzles each of which is positioned within the range 60-75% of the height of the reactor vessel.

13. A bioreactor according to claim 11 wherein all nozzles are distributed on 2 horizontal planes.

14. A bioreactor according to claim 12 wherein all nozzles are distributed on 3 horizontal planes.

15. A bioreactor according to claim 13, wherein each nozzle that shares a common horizontal plane has an angle to the horizontal or an angle to the radial direction that differs from an angle to the horizontal or an angle to the radial direction respectively of every other nozzle in the common horizontal plane.

16. A bioreactor according to claim 13, wherein an angle between each pair of neighbouring nozzles in the same horizontal plane is within the range: (360/number of nozzles in the plane)+/−10°.

17. A bioreactor according to claim 1, wherein at least one nozzle is moveable to provide an adjustable angle of injection to the horizontal or angle to the radial direction.

18. A bioreactor according to claim 1, wherein the circulation loop further comprises a cooling apparatus for the cooling reaction fluid from the reactor vessel.

19. A bioreactor according to claim 18, wherein the cooling apparatus comprises a gas inlet for supplying gas to the reaction fluid during cooling.

20. A bioreactor according to claim 1, wherein the reactor vessel is configured to operate with a headspace in the range of from 10 to 50% of the total volume of the reactor vessel.

21. A bioreactor according to claim 20, wherein all of the nozzles are located outside of the headspace, below a nominal fill level of the reactor vessel.

22. A bioreactor according to claim 1, wherein the reactor vessel does not comprise a mechanical agitator.

23. A bioreactor according to claim 11, wherein at least one nozzle is a two phase nozzle.

24. A bioreactor according to claim 1, wherein the reactor vessel further comprises a gas sparger.

25. A method of mixing fluids in a bioreactor according to claim 1, the method comprising the steps of:
withdrawing a liquid through the at least one outlet of the reactor vessel,
circulating the liquid through the circulation loop to the nozzle(s),
injecting the liquid through the nozzle(s) into the reactor vessel.

26. A method according to claim 25, wherein a loop flow ratio is at least 5 times per hour.

27. A method according to claim 25, wherein each nozzle has a nozzle speed in the range of 5 to 50 m/s.

28. A method according to claim 25, further comprising the step of cooling the reaction fluid as the reaction fluid passes from the reactor vessel outlet to the nozzles.

29. A method according to claim 28, wherein a gas is added to the reaction fluid as the reaction fluid is cooled.
